(19) United States
(12) Patent Application Publication
(10) Pub. No.: US 2017/0151288 A1
(43) Pub. Date: Jun. 1, 2017

(54) CELL THERAPY PREPARATION INDUCING THE RELEASE OF CELLULAR SUBSTANCES FOR TISSUE REGENERATION

(71) Applicant: Saewha JEON, Seoul (KR)

(72) Inventors: Saewha JEON, Seoul (KR); Yun Hee KIM, Seoul (KR); Hankyu JANG, Incheon (KR)

(21) Appl. No.: 15/319,825

(22) PCT Filed: Jun. 18, 2015
(86) PCT No.: PCT/KR2015/006171

§ 371 Date: Dec. 19, 2016

(30) Foreign Application Priority Data
Jun. 18, 2014 (KR) 10-2014-0074101

(51) Int. Cl. A61K 35/36

(52) U.S. Cl. CPC A61K 35/36

(57) ABSTRACT

Provided is a tissue regeneration material release-inducing cell therapeutic composition including a medium for cryopreservation of an animal cell which contains a protein, a sugar, a buffer, and a basal medium and does not contain DMSO, glycerol, and serum. The cell therapeutic composition of the present invention is characterized in that both an animal cell and the medium for cryopreservation of an animal cell have tissue regeneration efficacy, as a tissue regeneration accelerating material within cells is released into the medium for cryopreservation through freezing and thawing processes of the cells. In addition, the cell therapeutic composition of the present invention may be directly cryopreserved in a vial, ampoule or pre-filled syringe, thereby being highly convenient for use, and does not use a cryopreservative agent and serum and thus may be directly applied to a lesion without a separate washing process.

### TECHNICAL FIELD

The present invention relates to a cell therapeutic agent including a medium for cryopreservation of an animal cell and an animal cell.

### BACKGROUND ART

A medium for cryopreservation of an animal cell, used in cryopreservation of a cell therapeutic agent is generally dimethyl sulfoxide (DMSO), glycerol, ethylene glycerol (EG), or the like, and such medium is used additionally in combination with dextran, glucose, sucrose, mannitol, sorbitol, fructose, trehalose, raffinose, or the like according to purpose. However, mixing conditions according to purpose are complicated, manufacturing methods are complicated, and manufacturing costs are high. In addition, when DMSO is used as a medium for cryopreservation of an animal cell, DMSO has cytotoxicity and thus a washing process for removing DMSO when thawing after freezing is required. In addition, to increase cell viability, fetal bovine serum (FBS) is mainly used in an amount equal to or greater than that of a cryopreservative agent used, but must be finally removed because the FBS causes immune problems of heterologous proteins in a cell therapeutic agent. In addition, existing agents for external application are used in a spraying manner using a separate gas pressure-using apparatus, and thus, manufacturing costs are high and it is inconvenient to use. Therefore, there is a need to develop a cell therapeutic agent prepared by simple preparation processes and directly applicable to lesions without a separate washing process.

### DETAILED DESCRIPTION OF THE INVENTION

### Technical Problem

It is one object of the present invention to provide a tissue regeneration material release-inducing cell therapeutic composition including a medium for cryopreservation and an animal cell.

It is another object of the present invention to provide a method of preparing a tissue regeneration material release-inducing cell therapeutic composition including a medium for cryopreservation and an animal cell.

It is still another object of the present invention to provide a cell therapeutic agent from which a tissue regeneration accelerating material is released by freezing.

### Technical Solution

Unless defined otherwise, all technical and scientific terms used herein have the same meaning as commonly understood by those of ordinary skill in the art to which the present invention pertains. In general, the nomenclature used herein is well known and commonly used in the art.

Hereinafter, the present invention will be described in detail. The present invention provides a tissue regeneration material release-inducing cell therapeutic composition including: a medium for cryopreservation of an animal cell which includes a protein, a sugar, an amino acid, a buffer, and a basal medium and does not include DMSO, glycerol, ethylene glycerol, and serum which are conventional cryopreservative agents; and an animal cell.

The term “cell therapeutic agent” as used herein refers to a therapeutic agent using autologous, allogenic or xenogenic cells to recover the function of a tissue and, in the present invention, refers to a therapeutic agent used to regenerate an injured tissue by using an animal cell.

The term “medium for cryopreservation” as used herein refers to a culture that, during cryopreservation of an animal cell, preserves the cell so that the size and shape thereof are constantly maintained during freezing and thawing. In addition, the medium for cryopreservation according to the present invention does not include DMSO, glycerol, ethylene glycerol, and serum which are conventional cryopreservative agents, and thus, a separate washing process is not required.

The term “tissue regeneration material” as used herein refers to a protein such as a cytokine or the like released by an animal cell. In particular, according to the present invention, the cell therapeutic agent releases a protein such as a cytokine or the like in an animal cell into the medium for cryopreservation through freezing and thawing processes of the cell. Thus, a separate time for release of a cytokine is not required, and an effect of accelerating initial wound healing may be obtained by previously released cytokine. Therefore, the present invention provides a tissue regeneration material release-inducing cell therapeutic agent, and a cytokine included in the cell therapeutic agent of the present invention is released into a lesion by biological metabolism, thereby regenerating the corresponding tissue.

Various factors that may affect cell freezing include cell type, cell size, cell concentration, temperature, medium composition, pH, osmotic pressure, and the like, but medium composition during freezing is the most important factor.

In an embodiment of the present invention, a protein in the medium composition may be included in an amount of 1 wt % to 50 wt % based on a total weight of the medium. When the amount of the protein is less than 1 wt %, effects during freezing are insignificant. On the other hand, when the amount of the protein is greater than 50 wt %, it may affect an experiment for measuring useful materials included in cells.

In addition, the medium for cryopreservation may further include at least one of a sugar and a vitamin. The amount of the sugar may range from 0.1 wt % to 20 wt % based on a total weight of the medium composition. When the amount of the sugar is less than 0.1 wt %, freezing effects deteriorate. On the other hand, when the amount of the sugar is greater than 20 wt %, the viscosity of the sugar is increased and thus it is not easy to spray the cell therapeutic agent.

In addition, the buffer may be included in an amount of 0.01 wt % to 10 wt % based on a total weight of the medium composition. When the amount of the buffer is less than 0.01 wt %, a buffer effect is insignificant at appropriate pH. On the other hand, when the amount of the buffer is greater than 10 wt %, the buffer may have cytotoxicity according to type thereof.

In an embodiment of the present invention, the protein may be one selected from plasma proteins such as albumin; cell structural proteins such as actin and keratin; extracellular matrix proteins such as collagen, elastin, and fibronectin; cell adhesion proteins such as integrins, cadherins, and selectins; growth factors such as transforming growth factors (TGFs) and fibroblast growth factors (FGFs); hormones such as insulin and estrogen; amino acids such as L-alanine, L-arginine, L-cysteine, L-glutamine, and L-lysine; and mixtures thereof, but the present invention is not limited to the above examples.

In an embodiment of the present invention, the protein may be most preferably albumin. Albumin is a protein that makes up the largest portion of blood plasma, and may act as a carrier protein of various small molecules or may be used as a cryopreservative agent due to its effect of helping cell growth in cell culture. In addition, albumin may replace bovine serum commonly used in cell freezing and thus may enhance safety as a drug and have an effect on maintaining the stability of various proteins released from cells.

In an embodiment of the present invention, the sugar may include monosaccharides, disaccharides, and polysaccharides. Examples of suitable sugars include dextrose, maltose, glucose, lactose, sucrose, trehalose, mannose, maltose, raffinose, cellobiose, gentiobiose, isomaltose, arabinose, fructose, melezitose, melibiose, sorbitol, triose, xylose, mannitol, and sorbitol, but the present invention is not limited to the above examples.

In an embodiment of the present invention, the sugar may be sucrose, raffinose, and/or dextrose. Sucrose has been reported to have a higher cryopreservation effect than that of glycerol, is useful for long-term preservation of cells, and, in particular, may be used for cryopreservation of red blood cells. Raffinose may be used for, in particular, sperm freezing, and dextrose may be preferably used for cryopreservation of red blood cells.

In an embodiment of the present invention, the vitamin may be selected from L-ascorbic acid, folic acid, i-inositol, vitamin B12, and mixtures thereof, but the present invention is not limited thereto.

In addition, the medium for cryopreservation of an animal cell includes a buffer. The buffer of the present invention protects cells and thus may constantly maintain the size and shape thereof during freezing and thawing and improve an ability to endure storage. In an embodiment of the present invention, the buffer may be selected from HEPES, Hank's Balanced Salt Solution (HBSS), Earle's Balanced Salt Solution (EBSS), tricine, tris, TES, PIPES, sodium citrate, sodium acetate, sodium phosphate, sodium β-glycerophosphate, triethanolamine, sodium bicarbonate, sodium chloride, potassium chloride, calcium chloride, and mixtures thereof, but the present invention is not limited thereto.

In addition, the medium for cryopreservation of an animal cell, according to the present invention includes a basal medium. The term “culture medium” as used herein refers to a composition including essential components needed for the growth and proliferation of cells in vitro. The medium of the present invention is characterized in that a basal medium not including DMSO, glycerol, ethylene glycerol, and serum which are cryopreservative agents is used. The basal medium may be an artificially synthesized medium or a commercially prepared medium. The commercially prepared medium may be, for example, Dulbecco's Modified Eagle's Medium (DMEM), Minimal Essential Medium (MEM), Basal Medium Eagle (BME), RPMI 1640, F-10, F-12, α-Mineral Essential Medium (α-MEM) (available from Gibco, Invitrogen, New York.), Glasgow's Mineral Essential Medium (G-MEM),

Iscove's Modified Dulbecco's Medium (IMDM), AmnioMax, AmnioMax□ complete Medium (available from Gibco, New York, USA), Chang's Medium MesemCult-XF Medium (available from STEMCELL Technologies, Vancouver, Canada), McCoy's 5A, RPMI1640, Williams' medium E, or Iscove's Modified Dulbecco's Medium (IMDM), but the present invention is not limited thereto.

When the glycerol or DMSO, which is cell permeable, is used as a medium for cryopreservation, the glycerol or DMSO permeates into cells and thus decreases water content in the cells, thereby suppressing formation of ice crystals during freezing. However, the medium for cryopreservation according to the present invention includes a sugar such as sucrose, raffinose, or dextrose, serum, and albumin, which are cell non-permeable materials. Thus, the medium prevents the discharge of water from cells right before freezing to thus control osmotic equilibrium, thereby preventing damage to a cell membrane, and has no cytotoxicity as compared to glycerol and DMSO which are cell-permeable cryopreservative agents.

In addition, the medium for cryopreservation of an animal cell according to the present invention is characterized in that a useful material such as a cytokine in an animal cell is released into the medium for cryopreservation through a freezing process and thus the medium itself as well as an animal cell may heal a wound and regenerate a tissue.

In an embodiment of the present invention, the animal cell may have a cell concentration of 1×10⁵ cells/ml to 1×10⁸ cells/ml in the medium, preferably a cell concentration of 1×10⁶ cells/ml to 1×10⁷ cells/ml in the medium.

In an embodiment of the present invention, the animal cell may be an adult cell or a stem cell.

In an embodiment of the present invention, the adult cell is present in the epidermis, dermis and subcutaneous fatty layer and may be selected from the group consisting of a keratinocyte, a melanocyte, a Langerhans cell, a Merkel cell, a fibroblast, a vascular endothelial cell, an adipocyte, a hair follicle stem cell, a hemocyte, a hepatocyte, a nerve cell, a ligament cell, an epithelial cell, a cartilage cell, an osteocyte, and mixtures thereof, but the present invention is not limited thereto. In an embodiment of the present invention, as a source tissue of the keratinocyte, differentiated skin, a skin appendage or an embryonic stem cell and an induced pluripotent stem cell (iPS cell) are suitable. When the source tissue is skin, the skin may be preferably derived from the foreskin, armpit, hip, breast, scalp, pubic region, or scrotum. When the source tissue is a skin appendage, the skin appendage may be derived from a hair follicle, a sweat gland, a sebaceous gland, or a capillary vessel. In this regard, a hair derived from an anagen hair follicle and having keratinocytes of the hair follicle, attached thereto may be preferably used.

In an embodiment of the present invention, the stem cell may be selected from the group consisting of an embryonic stem cell, an adult stem cell, an induced pluripotent stem cell (iPS cell), and mixtures thereof, but the present invention is not limited thereto. The adult stem cell may be separated from various tissues and includes, for example, placenta-derived stem cells, bone marrow-derived stem cells, cord blood-derived stem cells, adipose-derived stem cells, stillborn fetal brain-derived neural stem cells, adult cell-derived mesenchymal stem cells, or the like.

In an embodiment of the present invention, the cell therapeutic agent may be used for the regeneration of tissues selected from skin, cartilage, bone, blood vessels, the brain, the liver, the heart, ligaments, muscles, the spinal cord, blood, bone marrow, the lungs, teeth, nerves, corneas, retinas, the esophagus, the spine, kidneys, the pancreas, and the urethra, but the present invention is not limited thereto.

The present invention also provides a method of preparing a tissue regeneration material release-inducing cell therapeutic composition, including: preparing a medium for cryopreservation of an animal cell by mixing a protein and a buffer with a basal medium; introducing an animal cell into the medium for cryopreservation of an animal cell; and freezing the medium for cryopreservation with the animal cell introduced thereinto.

The present invention also provides a vial, ampoule or pre-filled syringe in which the cell therapeutic agent is cryopreserved.

The term “pre-filled syringe” as used herein refers to a syringe previously and directly filled with a drug and, in the present invention, refers to a ready-to-use syringe previously filled with the cell therapeutic agent and in which the therapeutic agent is preserved. The pre-filled syringe according to the present invention is filled with the cell therapeutic agent and may be thawed after cryopreservation and then directly applied to a lesion to regenerate the corresponding tissue without a separate washing process. In addition, the cell therapeutic agent according to the present invention may be cryopreserved in a vial or an ampoule instead of the pre-filled syringe and then may be applied via a syringe right before application thereof to a lesion.

The cell therapeutic agent according to the present invention is a ready-to-use cell therapeutic agent prepared such that a syringe is filled with cells and the cells are cryopreserved. A conventional cell therapeutic agent has a principle in which a wound is healed by a cytokine secreted through processes such as cell engraftment, cell division, cell proliferation, cell migration, cell differentiation, and the like. Thus, a certain period of time is required from application of the cell therapeutic agent to a lesion to secretion of a protein such as a cytokine or the like.

Unlike this, the medium for cryopreservation according to the present invention does not include DMSO, glycerol, ethylene glycerol, and serum, and thus, a separate washing process is not required. In addition, in the cell therapeutic agent of the present invention, a protein such as a cytokine or the like in an animal cell is partially released into the medium for cryopreservation through cell freezing and thawing processes. Thus, a separate time for the release of a cytokine is not required, and an effect of accelerating initial wound healing is obtained by the previously released cytokine. That is, the cell therapeutic agent of the present invention has wound healing efficacy for both an animal cell and a medium for cryopreservation. Thus, the cell therapeutic agent may be cryopreserved after a pre-filled syringe is filled therewith and may be directly applied to a lesion after thawing thereof. That is, the cryopreserved cell therapeutic agent of the present invention may be directly used after a thawing process without washing. Thus, a cytokine included in the cell therapeutic agent according to the present invention is released by biological metabolism in a lesion to thus regenerate the corresponding tissue.

### Advantageous Effects

In a cell therapeutic agent according to the present invention, a protein such as a cytokine or the like inside a cell is partially released into a medium for cryopreservation of an animal cell through cell freezing and thawing processes and thus the cell therapeutic agent can directly accelerate wound healing when applied to a wound. The cell therapeutic agent according to the present invention has wound healing efficacy for both an animal cell and a medium for cryopreservation and thus initial wound healing by the released cytokine may be accelerated. In addition, the cell therapeutic agent according to the present invention does not include DMSO, glycerol, ethylene glycerol, and serum which are cryopreservative agents and thus may be directly applied to a lesion without a separate washing process.

### DESCRIPTION OF DRAWINGS

FIG. 1 illustrates a cell therapeutic agent with which a pre-filled syringe is filled, according to the present invention.

FIG. 2 illustrates analysis results of colony-forming efficiency of keratinocytes of pipette type (A) as a control and pre-filled syringe type cell therapeutic agent according to the present invention (B).

FIG. 3 illustrates quantitative results of released amounts of cytokines such as Interleukin 1 alpha (IL-1α), fibroblast growth factor (FGF), transforming growth factor alpha (TGF-α), and vascular endothelial growth factor (VEGF) of cell lysates and cell-free supernatants (CFSs) of a cell and an animal cell after a freezing process.

FIG. 4 illustrates results of a 3 day-long analysis of an effect of a CFS on proliferation (A) and cell migration (B) of keratinocytes to analyze wound healing of the CFS (CON: negative control, EGF: positive control, and CFS: cell-free supernatant).

FIG. 5 illustrates results of comparison between changes in lesions of wound-induced imprinting control region (ICR) mice with no treatment as a negative control and wound-induced mice treated with the cell therapeutic agent according to the present invention on days 4, 7, 10, and 14.

FIG. 6 is a graph showing wound healing of wound-induced ICR mice with no treatment as a negative control and wound-induced mice treated with the cell therapeutic agent according to the present invention on days 4, 7, 10, and 14.

### BEST MODE

Hereinafter, the present invention will be described in further detail with reference to the following examples. It will be obvious to one of ordinary skill in the art to which the present invention pertains that these examples are provided only for illustrative purposes and are not intended to limit the scope of the present invention.

### Example 1

### Culturing and Freezing of Keratinocytes

### Example 1-1

### Culturing of Keratinocytes

Keratinocytes derived from human foreskin was co-cultured with 3T3 feeder by using a DMEM:F12(3:1) medium containing 10% FBS. Thereafter, subcultured keratinocytes were cultured in a T75 culture flask and the 3T3 feeder was removed therefrom on 5 to 6 days after the culturing, and then only the keratinocytes were collected.

### Example 1-2

### Preparation of Medium for Cryopreservation of Animal Cell and Freezing of Keratinocytes

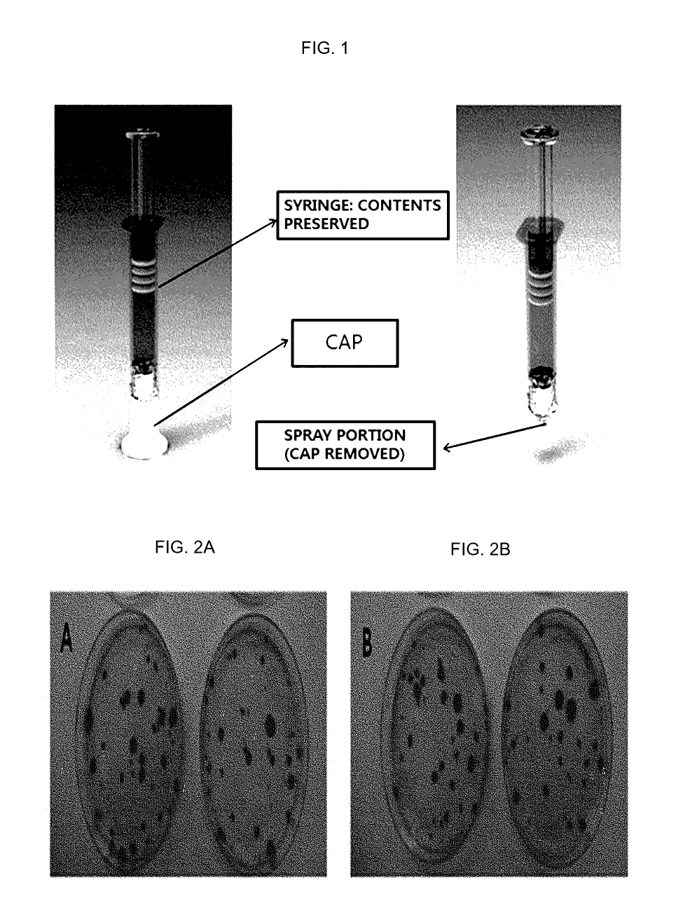

A medium for cryopreservation of an animal cell was prepared by mixing 1% human albumin, 1% sucrose, 1% dextrose, 1% raffinose, 25 mM HEPES, and 20 mM sodium carbonate (NaHCO₃) in a DMEM(GIBCO BRL) medium. 2×10⁶ cells/ml of keratinocytes were mixed in the medium. 1 ml of the mixed cell solution was transferred to respective pre-filled syringes for pre-filling, followed by cryopreservation thereof by gradually reducing the temperature from −20° C. to −70° C. (see FIG. 1).

### Example 2

### Analysis of Colony-Forming Efficiency

The cryopreserved cells were thawed and then sprayed using the pre-filled syringes to analyze a colony-forming efficiency. A control using a general pipette and the pre-filled syringe type cell therapeutic agent according to the present invention were compared and analyzed. The cryopreserved cells were sprayed into a culture vessel so that the number of total cells was 1×10² cells and then the resulting cells were co-cultured with 3T3 feeder in a DMEM/F12 medium containing 10% FBS for 12 days. On day 12 after the culturing, the resulting cells were fixed in 10% formalin and then stained using a rhodamine B dye solution.

Similar to the method of using a pipette, the keratinocytes sprayed through the pre-filled syringe were uniformly attached to the culture vessel and, when 1×10² of keratinocytes were sprayed, the colony-forming efficiency of the keratinocytes sprayed through the pre-filled syringe on day 12 after the culturing was observed as equal to or greater than that of the case of using the pipette (see FIG. 2).

### Example 3

### Cytokine Analysis of Keratinocytes Cryopreserved in Pre-Filled Syringe

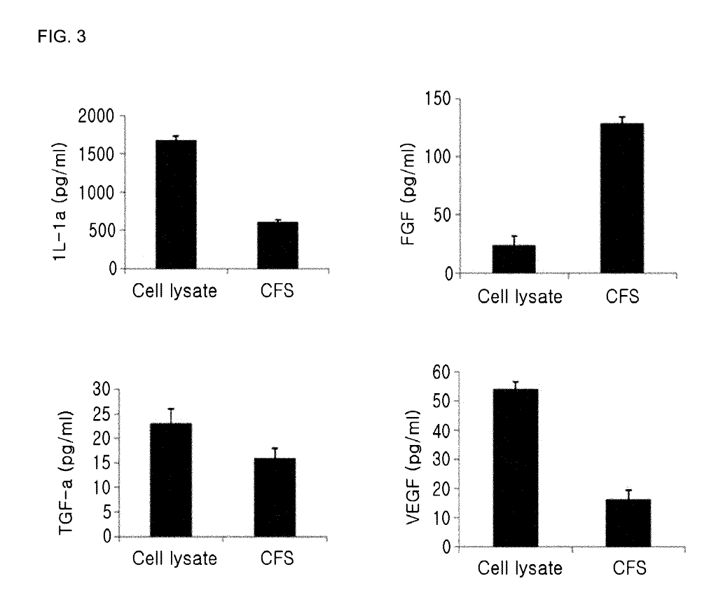

To measure the amounts of cytokines released into a cell lysate obtained by lysing the cells cryopreserved in a pre-filled syringe and a medium for cryopreservation, the cryopreserved cells were centrifuged to separate the cells and a medium supernatant (a cell-free supernatant (CFS)). As a result of quantification of cytokines involved in wound healing by an enzyme-linked immunosorbent assay (ELISA) method, IL-1α, FGF, VEGF, and TGF-α were measured in both the cells and the medium for cryopreservation (see FIG. 3).

### Example 4

### Analysis of In Vitro Wound Healing of CFS

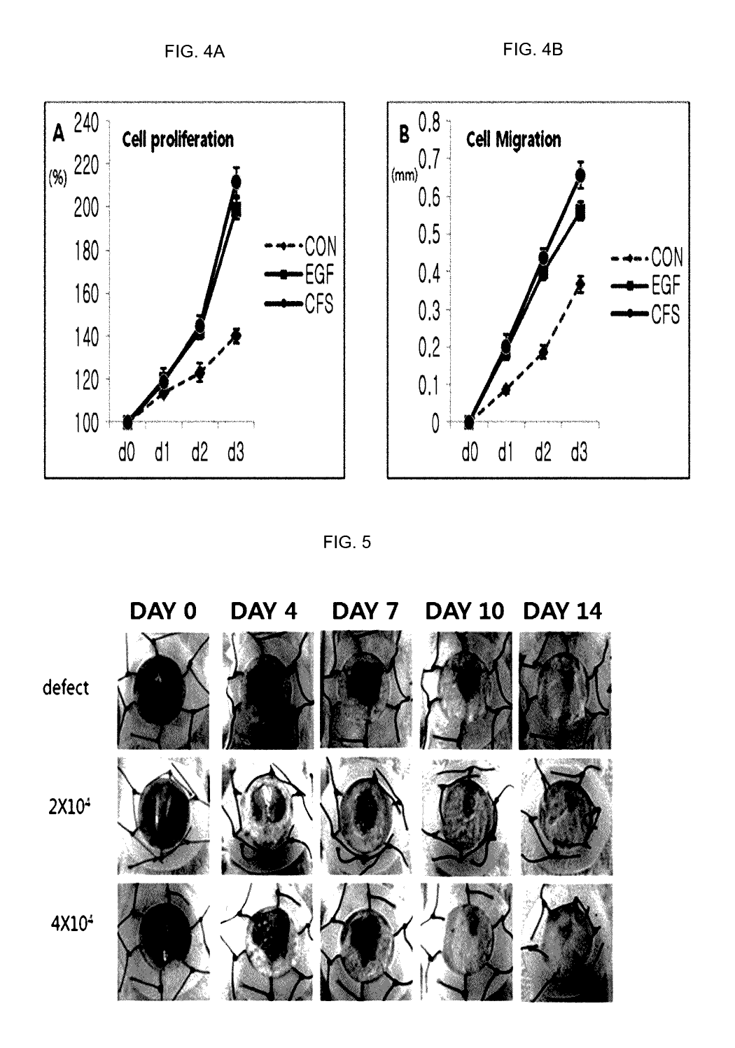

To analyze wound healing of a CFS, an effect of the CFS on proliferation and migration of keratinocytes, which is a main mechanism of re-epithelialization, was analyzed. The cells cryopreserved in a pre-filled syringe were centrifuged to separate only a supernatant. The proliferation of keratinocytes was analyzed by an MTT assay, the cell migration was analyzed by a megacolony assay, 1 ng/ml EGF was used as a positive control, and the analysis was performed for 3 days. As a result of analysis, an increase to 140% in cell proliferation (see FIG. 4A) was observed in a negative control (CON) on day 3 after culture, while a CFS-treated group exhibited an increase to 212% in cell proliferation on day 3 after culture and had similar efficacy to the positive control (EGF). An increase of cell migration (see FIG. 4B) of the CFS-treated group was 1.8 times that of the negative control (CON) on day 3 after culture and the CFS-treated group had similar efficacy to the positive control (EGF).

### Example 5

### Analysis of In Vivo Wound Healing of Keratinocytes Cryopreserved in Pre-Filled Syringe

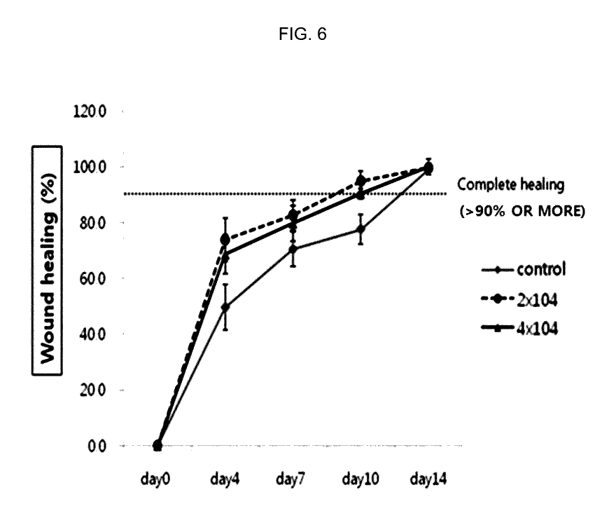

Hair on a back part of each of 24 ICR mice were removed, a wound having a diameter of 1.2 cm was induced on a center of the skin by using a sample punch, and keratinocytes cryopreserved in a pre-filled syringe were thawed at room temperature for several minutes and then sprayed onto the skin so that 2×10⁴ keratinocytes or 4×10⁴ keratinocytes were applied thereon. Thereafter, each wound site was covered with a Vaseline gauze and a foam dressing (Mepilex, Safetac), then affixed using a Peha-haft® bandage (manufactured by HARTMANN), and the wound sites were photographed on days 4, 7, 10, and 14. Wound healing was obtained by measuring an area of re-epithelialization on each date with respect to an area of re-epithelialization of the wound at an initial stage and representing the measured areas as percentage. In the case of application of 2×10⁴ keratinocytes or 4×10⁴ keratinocytes, 90% or more of wound healing, i.e., complete healing (see FIGS. 5 and 6) was observed in the keratinocyte-treated group on day 10 after the wound was induced, as compared to a negative control (defect) with no treatment after the wound was induced.

1. A tissue regeneration material release-inducing cell therapeutic composition comprising: a medium for cryopreservation of an animal cell, comprising a protein, a sugar, a buffer, and a basal medium; and an animal cell.

2. The tissue regeneration material release-inducing cell therapeutic composition of claim 1, wherein the protein comprises one selected from albumin, actin, keratin, collagen, elastin, fibronectin, integrins, cadherins, selectins, transforming growth factors (TGFs), fibroblast growth factors (FGFs), insulin, estrogen, and mixtures thereof.

3. The tissue regeneration material release-inducing cell therapeutic composition of claim 1, wherein the sugar comprises one selected from dextrose, maltose, glucose, lactose, sucrose, trehalose, mannose, raffinose, cellobiose, gentiobiose, isomaltose, arabinose, fructose, melezitose, melibiose, sorbitol, triose, and mixtures thereof.

4. The tissue regeneration material release-inducing cell therapeutic composition of claim 1, wherein the buffer comprises one selected from HEPES, Hank's Balanced Salt Solution (HBSS), Earle's Balanced Salt Solution (EBSS), tricine, tris, TES, PIPES, sodium citrate, sodium acetate, sodium phosphate, sodium β-glycerophosphate, triethanolamine, sodium bicarbonate, sodium chloride, potassium chloride, calcium chloride, and mixtures thereof.

5. The tissue regeneration material release-inducing cell therapeutic composition of claim 1, wherein the basal medium comprises one selected from Dulbecco's Modified Eagle's Medium (DMEM), Minimal Essential Medium (MEM), Basal Medium Eagle (BME), F-10, F-12, α-Mineral Essential Medium (α-MEM), Glasgow's Mineral Essential Medium (G-MEM), DMEM:F12, McCoy's 5A, RPMI 1640, Williams' medium E, Iscove's Modified Dulbecco's Medium (IMDM), and mixtures thereof.

6. The tissue regeneration material release-inducing cell therapeutic composition of claim 1, wherein the animal cell is either an adult cell or a stem cell.

7. The tissue regeneration material release-inducing cell therapeutic composition of claim 6, wherein the adult cell comprises one selected from the group consisting of a keratinocyte, a melanocyte, a Langerhans cell, a Merkel cell, a fibroblast, a vascular endothelial cell, an adipocyte, a hair follicle stem cell, a hemocyte, a hepatocyte, a nerve cell, a ligament cell, an epithelial cell, a cartilage cell, an osteocyte, and mixtures thereof.

8. The tissue regeneration material release-inducing cell therapeutic composition of claim 6, wherein the stem cell comprises one selected from the group consisting of an embryonic stem cell, an adult stem cell, an induced pluripotent stem cell (iPS cell), and mixtures thereof.

9. The tissue regeneration material release-inducing cell therapeutic composition of claim 1, wherein the tissue is selected from skin, cartilage, bone, blood vessels, brain, liver, heart, ligaments, muscles, spinal cord, blood, bone marrow, lungs, teeth, nerves, corneas, retinas, esophagus, spine, kidneys, pancreas, and urethra.

10. A method of preparing a tissue regeneration material release-inducing cell therapeutic composition, the method comprising:
preparing a medium for cryopreservation of an animal cell by mixing a protein, a sugar, and a buffer with a basal medium;
introducing an animal cell into the medium; and
freezing the medium with the animal cell introduced thereinto.

11. A method of using a tissue regeneration material release-inducing cell therapeutic composition, wherein the cell therapeutic composition of claim 10 in a cryopreserved state is thawed and then directly used without washing.

12. A vial, ampoule or pre-filled syringe with the cell therapeutic composition according to claim 1cryopreserved therein.

13. A vial, ampoule or pre-filled syringe with the cell therapeutic composition according to claim 2 cryopreserved therein.

14. A vial, ampoule or pre-filled syringe with the cell therapeutic composition according to claim 3 cryopreserved therein.

15. A vial, ampoule or pre-filled syringe with the cell therapeutic composition according to claim 4 cryopreserved therein.

16. A vial, ampoule or pre-filled syringe with the cell therapeutic composition according to claim 5 cryopreserved therein.

17. A vial, ampoule or pre-filled syringe with the cell therapeutic composition according to claim 6 cryopreserved therein.

18. A vial, ampoule or pre-filled syringe with the cell therapeutic composition according to claim 7 cryopreserved therein.

19. A vial, ampoule or pre-filled syringe with the cell therapeutic composition according to claim 8 cryopreserved therein.

20. A vial, ampoule or pre-filled syringe with the cell therapeutic composition according to claim 9 cryopreserved therein.
